(19) United States
(12) Patent Application Publication
(10) Pub. No.: US 2026/0158263 A1
(43) Pub. Date: Jun. 11, 2026

(54) TRANSCUTANEOUS ENERGY TRANSMISSION SYSTEM FOR POWERING AN IMPLANT

(71) Applicant: FINEHEART, Pessac (FR)

(72) Inventors: Lionel CIMA, St Cyr sur Loire (FR); Arnaud MASCARELL, Montbazon (FR)

(21) Appl. No.: 19/150,102

(22) PCT Filed: Jan. 25, 2024
(86) PCT No.: PCT/EP2024/051830

§ 371 Date: Jul. 22, 2025

(30) Foreign Application Priority Data
Jan. 27, 2023 (FR) FR2300796

(51) Int. Cl. A61M 60/873; H01F 27/255; H01F 27/28; H01F 38/14; H02J 50/12

(52) U.S. Cl. CPC A61M 60/873; H02J 50/12; H01F 27/255; H01F 27/2804; H01F 38/14

(57) ABSTRACT

A transcutaneous energy transmission system for powering an implant, including a primary circuit with an inverter able to be powered by a DC voltage UpDC and a direct current IpDC, and a primary coil Lp able to be powered by the inverter, and a secondary circuit including a secondary coil Ls intended to be in magnetic coupling with the primary coil, a secondary capacitor Cs, a rectifier able to supply the implant with a DC voltage UsDC; the secondary circuit further including a switch IT for powering or not powering the rectifier. The system further includes a self-adaptive control circuit, connected to the primary circuit and configured to automatically control the DC voltage UpDC from the single measurements of UpDC and IpDC, and according to a constrained optimization algorithm.

The present invention relates to a Transcutaneous Energy Transfer System (TETS) for powering an implant.

This is a system for transferring energy without percutaneous connection, using a magnetic field.

The invention has a particularly interesting application in the field of pulsatile heart implants, but it can be applied to any type of device requiring non-contact energy transfer.

There are heart pumps that are pulsatile and synchronized to the native activity of the patient's heart, which is necessary to ensure certain vital physiological functions of the patient's cardiac system. For example, it is essential to reduce the rotation speed of the pump during diastole so as not to impede the filling of the systemic ventricle. The typical duration of a cardiac cycle varies between 300 ms and three seconds. Transition delays between diastole and systole are of the order of a few 10 ms.

As a result, consumption is itself pulsatile with an instantaneous power of the mechanical part of the pump which varies in a ratio of 1 to 10 between the different cardiac phases, all on time scales of the order of 100 ms.

This high intrinsic variability creates specific design constraints on the transcutaneous energy transmission device.

Documents U.S. Pat. Nos. 6,478,820 and 6,458,164 are known describing a conventional TET operating with minimalist implantable electronics and without the need for an external control loop in order to control the output voltage UsDC within a defined range.

The conventional TET comprises:
a primary circuit comprising an inverter able to be powered by a DC voltage UpDC and a direct current IpDC, a primary capacitor Cp and a primary coil Lp able to be powered by the inverter, and
a secondary circuit comprising a secondary coil Ls intended to be in magnetic coupling with the primary coil according to a magnetic coupling coefficient k, a secondary capacitor Cs, a rectifier able to supply the implant with a DC voltage UsDC; the secondary circuit further comprising a switch IT for powering or not powering the rectifier and a control circuit for the switch IT controlled from the local voltage UsDC. “Local” is understood to mean a voltage of the secondary circuit.

The benefit of the “conventional TET” solution relates to its reliability: no need for an external radiofrequency feedback loop, very few implantable components, and perfect adaptation to the implant constraints: low temperature rise when the coupling and/or the transmitted power is optimal. On the other hand, its sizing imposes a certain number of compromises which ultimately make the solution difficult to use in practice. Indeed, the solution works very well for a reduced power range and/or for a reduced magnetic coupling range, but said ranges are ultimately too small. In addition, the operating ranges between output power and magnetic coupling are interlinked. In the case of the “conventional TET”, as soon as the permissible output power range PoutDC is exceeded, either the overheating becomes prohibitive, or the device no longer functions at all beyond a certain coupling k. Conversely, as soon as the permissible coupling range k is exceeded, either the overheating becomes prohibitive, or the device no longer functions at all, having reduced its operating range in terms of output power PoutDC.

Finally, it is the combination of the high variability of k and PoutDC that leads to a reduction in the operating ranges of k and PoutDC, making the device unsuitable for use outside a laboratory or a clinical trial in a controlled environment. This is because the very performance and operation of the “conventional TET” depends non-linearly on both PoutDC and k. Thus, the optimal operating point of the system is different for each combination of PoutDC and k. In particular, it is possible to show that in the case of the “conventional TET”, this leads to a systematic over-dimensioning of the UpDC value. The downside is that the efficiency, overheating and usability (kmaxi and kmini as well as PoutDCmaxi and PoutDCmini) of the conventional TET are not optimal outside these narrow ranges, making them unsuitable for commercial use.

Also known are documents U.S. Pat. Nos. 9,855,376, 10,149,933, 11,235,141 and 11,534,225 describing the “conventional TET” with the addition of an AC current sensor and primary-side switching between two “on/off” modes based on the estimated state of the switch IT of the secondary circuit. This solution generates too much electromagnetic pollution and is not optimal from a performance point of view. This makes it unusable for a marketable product which has to comply with the requirements of standards to guarantee the safety of goods and people.

The aim of the present invention is to increase reliability by minimizing the number of components of the TETS.

Another aim of the invention is to increase the safety of goods and people by maintaining the voltage levels of distributed power supplies below so-called “very low voltage” thresholds and allowing a fault-free operating mode despite any loss of telecommunication between the implant and the external power supply device.

Another aim of the invention is to increase the usability of the TETS by extending the operating ranges of the coupling coefficient k and the output power PoutDC.

Another aim of the invention is to increase the efficiency, reduce the electromagnetic pollution, reduce the increase in temperature in living tissues, and reduce the dimensions and mass of the coils.

Another aim of the invention is to increase the level of integration of implantable electronics, thus facilitating the implantation thereof in the body of the patient.

At least one of the objectives is achieved with a transcutaneous energy transmission system for powering an implant, said system comprising:
a primary circuit comprising an inverter able to be powered by a DC voltage UpDC and a direct current IpDC, and a primary coil Lp able to be powered by the inverter,
a secondary circuit comprising a secondary coil Ls intended to be in magnetic coupling with the primary coil according to a magnetic coupling coefficient k, a secondary capacitor Cs, a rectifier able to supply the implant with a DC voltage UsDC; the secondary circuit further comprising a switch IT for powering or not powering the rectifier and a control circuit for the switch IT controlled locally from the voltage UsDC.

According to the invention, the system further comprises:
a self-adaptive control circuit, connected to the primary circuit and configured to automatically control the DC voltage UpDC from the single measurements of UpDC and IpDC, and according to a constrained optimization algorithm.

The control consists of varying the voltage UpDC continuously or discretely. This variation is a function of at least one predetermined constraint.

The invention is particularly noteworthy in that the only measurements made are those of UpDC and IpDC, and they are local, within the optimized primary circuit (optimized by the presence of the self-adaptive control circuit). No measurements come from the secondary circuit 2b, or even from the primary circuit 2a, and still less from a wireless communication link as is the case with the induction charging systems currently on the market.

Generally speaking, the primary circuit and the control circuit are intended to be arranged on the exterior of a patient, while the secondary circuit is intended to be arranged inside the patient in connection with the implant. The latter may be for example an electromechanical cardiac assistance device placed entirely or not in the heart of the patient.

With the invention, the performance and the use of the transcutaneous energy transmission system are optimized by automatically adapting the DC voltage UpDC based on variations in the consumption PoutDC(t) and/or the coupling coefficient k. Indeed, variations in the consumption PoutDC(t) and the coupling coefficient k modify the system performance variables, as well as the optimum operating point defined by the DC voltage UpDC and the direct current IpDC. By estimating these system performance variables and by measuring the DC voltage UpDC and the direct current IpDC, a correction of the DC voltage UpDC may or may not be applied so as to tend towards an optimum.

It is the self-adaptive control circuit that comprises the algorithm needed to control the primary circuit from data obtained by the single measurement of variables UpDC and IpDC without the need to set up an ad hoc control circuit from the secondary circuit. There are several types of constrained optimization algorithms. The user can select one thereof based on one or more predetermined constraints.

The self-adaptive control circuit is powered by a battery or the mains supply.

The invention can notably be used to automatically minimize the power absorbed by the transcutaneous energy transmission system based on its environment, by precisely adjusting the value of the DC voltage UpDC of the supply line.

The present invention allows efficient operation without mandatory communication of data between the implantable part and the external part. The only mandatory link between the external part and the implantable part is the magnetic coupling between the two coils. No radiofrequency or wired feedback link, as in the prior art, is required.

Real-time self-adaptation of the DC voltage UpDC according to the invention optimizes the performance of the transcutaneous energy transmission system notably by increasing transfer efficiency, reducing the heating of living tissue, reducing power consumption and increasing the range of k values, despite high real-time variability in the coupling k and power PoutDC.

The presence of the self-adaptive control circuit makes it possible to advantageously replace the feedback loop of the prior art by eliminating causes of defects.

The automatic control of UpDC according to the present invention allows a reduction in the amplitude of the voltage at the terminals of the coils Ls and Lp, thus improving not only the efficiency but also the reliability and the safety for goods and people. Preferentially, overvoltages can be kept less than 70 V peak (very low voltage limit) in order to keep any leakage currents in the event of insulation failure below a critical threshold for users.

According to an advantageous feature of the invention, the self-adaptive control circuit can be configured to estimate system performance variables from measurements of UpDC and IpDC. The constrained optimization algorithm can be configured with at least one predetermined constraint and associated with one of the system performance variables.

Once again, an advantageous feature of the invention is that the system performance variables such as the power consumed by the load, the efficiency, the magnetic coupling coefficient and the power dissipated in the secondary are obtained in real time without having to rely on measurements from the secondary circuit.

According to one embodiment of the invention, said system performance variables may comprise at least one or more of the following instantaneous variables: a duration Toff during which the switch IT is closed, a duration Ton during which the switch IT is open, an instantaneous continuous power PinDC supplying the inverter, a continuous power PoutDC at the output of the secondary circuit, an energy transfer efficiency RendTETS, a power dissipation Pdiss in the system.

Other variables can be estimated, such as for example the magnetic coupling coefficient k.

The single measurements of DC voltage UpDC and direct current IpDC according to the invention are required to estimate real-time performance variables such as k, PinDC, PoutDC and energy transfer efficiency.

Toff is the duration during which the switch IT is closed (Off phase), so the power is not transferred from the source to the load of the implantable part.

Ton is the duration during which the switch IT is open, so the power is transferred from the external part to the load of the implantable part.

The invention notably makes it possible to compensate for movements of the patient when these result in relative displacements between the coils and thus a variation in the magnetic coupling coefficient k, by modulating the DC voltage UpDC in order to maintain it at an optimum value (for example in order to maximize power transfer efficiency). In addition, it is possible to define extreme values, a UpDCmax and a UpDCmin, for example at values of 15 V and 1 V respectively. A nominal value UpDCnom, between UpDCmax and UpDCmin can also be predefined by the user. The system can be started up notably by setting UpDC equal to UpDCnom, this voltage being selected (by calculation or by experience) by the user within a range of values that are sufficiently high to allow cyclic operation of the switch IT.

The transmission of power is only correct on the necessary (but not sufficient) condition that the switch IT closes regularly, which means that the duration Toff must be non-zero, or in other words that the duration Ton must be less than a certain TonMAX value.

According to an advantageous feature of the invention, said constraints may comprise a minimum duration Toff during which the switch IT is closed, and/or a maximum duration Ton during which the switch IT is open, and/or a minimum instantaneous power PinDC, and/or a maximum efficiency RendTETS, and/or a minimum power dissipation Pdiss.

Thus, the DC voltage UpDC is regularly adapted to each cycle in order to work towards an optimum that respects said constraints.

The constraint can therefore for example be to reduce the power dissipation Pdiss as much as possible, and in any case to keep it below a maximum value deemed acceptable, such that the increase in temperature in living tissue does not exceed the limits imposed by physiology and/or international standards. Typically, beyond a 4K increase in temperature, tissues become necrotic and the standards impose a limit of 2K for internal tissues.

Advantageously, the measurements and the estimated variables can be taken cyclically.

A cycle can be defined by a pair of open and closed states of the switch IT. The measurements of UpDC and IpDC can be taken during one cycle and the estimates during another cycle. The estimates can be based on averaging measurements taken over several cycles.

The secondary coil is conventionally wound with Litz wire in order to reduce the equivalent resistance when the frequency of the inverter is high. This winding is connected to the electronics via a connector.

According to an advantageous feature of the invention, the secondary coil is made from tracks of a printed circuit PCB, for example with multilayer conductors.

Indeed, the use of a printed circuit board (PCB) secondary coil with notably multilayer conductors reduces the value of the equivalent internal resistance of the system, which is the cause of overheating, while facilitating industrial production.

Advantageously, the switch IT can be integrated into the printed circuit PCB of the secondary coil. This thus reduces the total equivalent resistance by virtue in particular of the elimination of the connector between the coil and the electronics.

Indeed, the contribution of the series resistance of the circuit of the switch IT becomes very high if the switch is too far from the secondary coil. In order to limit this resistance, as well as the resistance of the primary circuit and thus limit the heating of the secondary circuit when the switch is closed, the invention provides for this switch to be placed in the immediate vicinity of the secondary coil, and ideally directly on the PCB. In this preferred configuration, all of the electronics of the secondary circuit can be placed on the PCB of the implantable coil. Thus, the connection between the transcutaneous energy transmission system and the rest of the implantable device can be made via a very low-voltage DC link, thus considerably improving patient safety in the event of accidental insulation failure.

According to one embodiment of the invention, a magnetic core can be preferentially placed in the secondary coil Ls or optionally in the primary coil Lp.

This thus improves the value of the secondary inductance Ls, which extends the range of k values by reducing the minimum value kmini for a given voltage UpDCmaxi.

Preferably, the magnetic core can be a superparamagnetic composite core.

This is a non-conductive core with substantially no hysteresis losses.

The superparamagnetic filler can be placed directly in the core of the planar windings, or else in the PCB, or else in the overmolding and/or protective material of the secondary coil.

According to the invention, the primary circuit can further comprise a primary capacitor Cp; the primary coil Lp and the primary capacitor Cp can have values so as to be in resonance when the coupling k between the primary coil and the secondary coil is below a predetermined coupling threshold. The coupling threshold can be equal to 1% or even 1/1000. In such a condition, we consider ourselves to be in a state of decoupling.

In addition, the secondary coil Ls and the secondary capacitor Cs can have values so as to be in resonance when the coupling k between the primary coil and the secondary coil is below a predetermined coupling threshold.

The primary and secondary circuits can thus be at resonance when the coupling is substantially zero, i.e. when k=0. Thus, this leads to overconsumption on the primary side in the event of decoupling, and thus to the detection of decoupling. This also means very low consumption (almost zero) on the primary side when the coils are coupled but the switch is closed.

The single analysis of the power PinDC(t) can automatically control the transcutaneous energy transmission system.

According to one embodiment of the invention, the transcutaneous energy transmission system may comprise a radio link able to connect the self-adaptive control circuit with the implant so as to retrieve real-time information from the implant.

This wireless radio link can be used to exchange real-time information such as power consumption in W, heart rate in Hz, QRS detection time, the actual value of PoutDC(t), etc., between the implant and the external part of the transcutaneous energy transmission system.

This optional feedback loop further improves the control but may not be available without limiting the overall operation of the transcutaneous energy transmission system.

This information can be used, for example, to anticipate changes in PoutDC(t) and thus optimize the values of UpDC. Typically, the value of UpDC can be modified upwards or downwards in larger increments, depending on the actual consumption estimate. Thus, this reduces the time taken for the system to converge towards its optimum operating point. This option is particularly useful for improving the dynamic properties of the system when the parameters k and PoutDC vary rapidly. In practice, variations in magnetic coupling are induced by the patient and are very slow, of the order of a second or even several seconds. The rate of change of the variable PoutDC depends on the charge and the capacity of the storage capacitor CStock placed in the rectifier. This rate of change is therefore a design parameter that can be taken into account when optimizing the convergence dynamics of the algorithm towards an optimum.

By way of example, the self-adaptive control circuit can be configured to adapt the operating frequency of the inverter to the resonance frequency of the secondary circuit based on information from the implant.

This adaptation can take place during a calibration and pairing step. The frequency value of the resonant circuit of the impact can be stored in a memory thereof, such that the external system can be exchanged and automatically adapted thereto.

According to one embodiment of the invention, the transcutaneous energy transmission system may comprise at least one pulse width modulator to control the inverter at a frequency less than 301 kHz, less than 151 kHz or less than 81 kHz.

Such low frequencies enable quasi-sinusoidal control by pulse width modulation (PWM) to be implemented, thus controlling the quality of the network and limiting harmonics, to reduce electromagnetic pollution (improve electromagnetic compatibility-EMC), and also reduce the skin effect in windings, and therefore heating losses.

The system according to the invention may comprise a precalculated pulse width modulator with one or more harmonic contents of predefined amplitude, frequency and phase.

These harmonic contents of the pulse width modulator can be precalculated to switch rapidly from one to the other, notably when decoupling has been detected, or else during the OFF phase. The triggering of this total or partial deactivation is then controlled by the self-adaptive control circuit.

Advantageously, the system according to the invention may comprise a watchdog to trigger an action when an instantaneous power PinDC supplying the inverter does not vary in intensity beyond a predetermined power threshold for a predetermined duration. The threshold can be set at a value of 30% between max power and min power. And the duration can be 5 seconds for example. This absence of variation is synonymous with stalling, i.e. the voltage UsDC is below the acceptable range.

In the event of stalling, the voltage UpDC must be increased to enable re-connection. There are several strategies which depend on the nature of the implant:
Slow increase in UpDC
Instant switch to UpDCMax
Instant switch to UpDCNominal
Etc.

According to one embodiment of the invention, the self-adaptive control circuit may comprise a DC voltage sensor, a direct current sensor and a processing unit.

The processing unit allows estimates to be made in real time over an observation period of typically 1 ms and periodically updated. This processing unit is also configured to run the constrained optimization algorithm.

According to another aspect of the invention, a method is proposed for controlling a DC voltage UpDC in a transcutaneous energy transmission system as defined in any one of the preceding systems, this method comprising, at each cycle defined by open and closed states of the switch IT, the following steps:
measuring the DC voltage UpDC and direct current IpDC,
estimating system performance variables, storing these variables, and comparing with values of these variables obtained during a previous cycle,
controlling the DC voltage upDC from the single measurements of UpDC and IpDC, and according to a constrained optimization algorithm.

Other benefits and features of the invention will become evident upon examining the detailed description of an entirely non-limiting implementation, and from the enclosed drawings wherein:

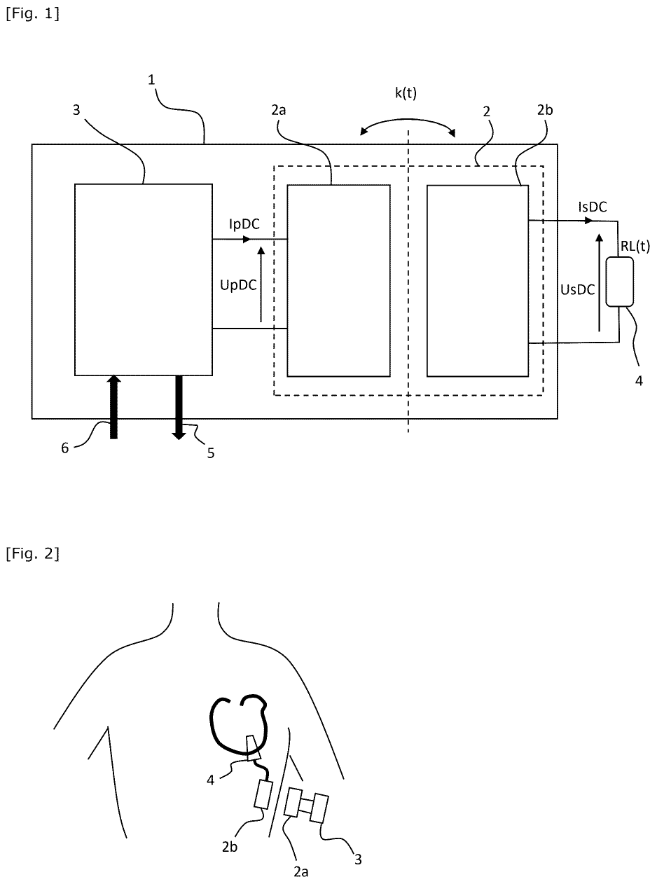

FIG. 1 is an overall schematic view of the transcutaneous energy transmission system,

FIG. 2 is a schematic view of the transcutaneous energy transmission system once installed on a patient,

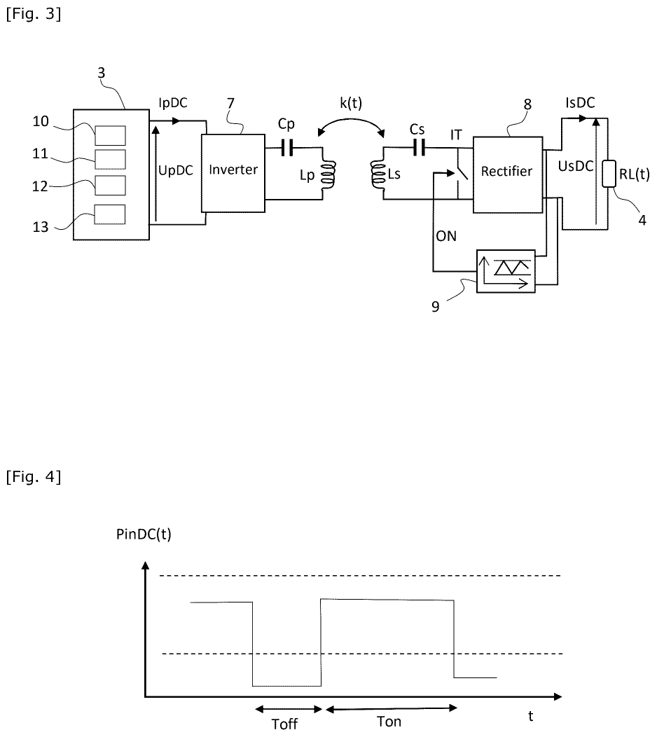

FIG. 3 is a more detailed schematic view of the transcutaneous energy transmission system,

FIG. 4 is a schematic view of a graph of the instantaneous power PinDC supplying the inverter of the transcutaneous energy transmission system based on time,

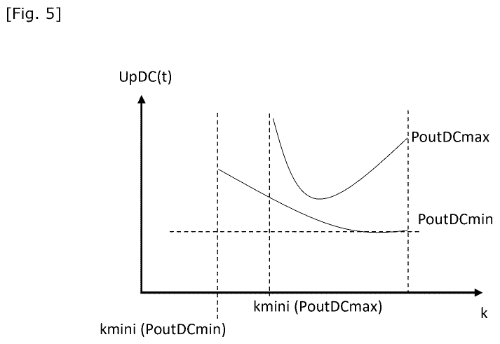

FIG. 5 is a schematic view of a graph of the voltage UpDC supplying the inverter of the transcutaneous energy transmission system based on the coupling coefficient k.

The embodiments which will be disclosed hereinafter are in no way limiting; in particular, it is possible to implement variants of the invention that comprise only a selection of the features disclosed hereinafter in isolation from the other features disclosed, if this selection of features is sufficient to confer a technical benefit or to differentiate the invention with respect to the prior art. This selection comprises at least one preferably functional feature which lacks structural details, or only has a portion of the structural details if that portion only is sufficient to confer a technical benefit or to differentiate the invention with respect to the prior state of the art.

In particular, all the variants and all the embodiments disclosed are intended to be combined with each other in any combination where there is no technical obstacle to doing so.

In the figures, the same reference has been used for the features that are common to several figures.

Although the invention is not limited thereto, a transcutaneous energy transmission system will now be described for powering an implant which is an intraventricular heart pump.

FIG. 1 generally shows the Transcutaneous Energy Transfer System 1 (TETS) according to the invention. TETS 1 comprises a non-optimized transcutaneous energy transmission subsystem 2, otherwise known as “conventional TET”, intended to power a resistor RL(t) representing the equivalent load of the implant and which may vary over time.

The transcutaneous energy transmission system TETS 1 according to the invention further comprises a control circuit 3 intended to power the conventional TET 2 with DC voltage UpDC and direct current IpDC.

The control circuit 3 comprises inputs/outputs for transmitting data to the user via an output 5, and optionally receiving external data via an input 6.

The conventional TET 2 consists of two circuits: a primary circuit 2a separate from a secondary circuit 2b. The two circuits, primary 2a and secondary 2b, are in magnetic coupling with a magnetic coupling coefficient k that can vary with time.

FIG. 2 is a schematic view of TETS 1 installed on a patient. The control circuit 3 and the primary circuit 2a are placed outside the body of the patient. The secondary circuit 2b is installed inside the patient and is intended to supply DC voltage to the implant 4 which is for example a pulsatile heart pump.

The function of the TETS 1 according to the invention is to self-adapt the DC voltage UpDC in real time in order to optimize the performance of the TETS 1 notably by increasing the transfer efficiency, reducing the heating of living tissue, reducing consumption, increasing the maximum operating value of k (kmaxi), reducing the minimum operating value of k (kmini) despite a high real-time variability of the coupling k and the power PoutDC at the output of the secondary circuit 2b.

TETS 1 also improves the accuracy of estimates of the system performance variables as will be seen later. These performance variables notably constitute the data that the control circuit can transmit to the user to indicate the quality level of the power transmission.

Advantageously, the conventional TET 2 is a transcutaneous energy transmission system without a control loop between an implantable part and an external part as described in documents U.S. Pat. Nos. 6,478,820 and 6,458,164.

More precisely and with reference to FIG. 3, the conventional TET 2 according to the invention is composed of:
an implantable secondary coil with self-inductance Ls, preferentially planar,
an external primary coil with self-inductance Lp, preferentially planar, magnetically coupled to the secondary coil Ls. The magnetic coupling k is defined by the value of the mutual inductance M between Ls and Lp, defined by M²=k²Lp. Ls such that 0≤k≤1. Magnetic coupling can vary over time, throughout its entire possible range [0;1],
external inverter-type power electronics 7 to convert the DC power PinDC into AC power at a fundamental frequency Fex. The primary circuit 2a can include a resonance capacitor Cp, preferentially placed in series (and/or in parallel) with the primary coil Lp and whose capacitance is selected such that the circuit (Lp,Cp) resonates at the frequency Fex, that is Fex.2.pi=1/root (Lp.Cp),
a secondary series-resonance capacitor Cs whose capacitance is selected such that the circuit (Ls, Cs) also resonates at the frequency Fex, that is Fex.2.pi=1/root (Ls.Cs),
a switch IT placed in parallel with the circuit (Ls, Cs), controlled by the ON signal,
internal rectifier-type power electronics 8 for converting AC power into DC power PoutDC(t). Preferentially, the rectifier 8 is a passive, single- or full-wave rectifier based on one or more diodes, preferably low-threshold Schottky diodes, and an energy storage capacitor Cstock and
a control circuit 9 self-powered by the rectifier 8 and allowing the ON signal to be generated from a measurement of the DC voltage UsDC (t) such that ON<=‘1’ if UsDC is increasing and/or rises above UsDCmax, then returns to ON<=‘0 if UsDC is decreasing and/or falls below UsDCmin (hysteresis regulation). UsDC is the mean value of the voltage UsDC (t).

The conventional TET 2 is connected to a load RL(t) whose characteristic may be variable over time, in particular pulsatile in the case of the heart pump for example.

The conventional TET 2 automatically transfers DC-DC energy, regulating the output voltage UsDC between the values UsDCmin and UsDCmax according to a Ton+Toff period cycle, automatically timed by the state of the switch IT. IT=1 (closed) during Toff time and IT=0 (open) during Ton time.

The conventional TET 2 used alone presents overheating and performance problems. The invention is particularly noteworthy in that it introduces the control circuit 3 for adjusting the value of the DC supply voltage of the primary circuit 2a without recourse, in a fundamental way, to feedback from the secondary circuit 2b.

In FIG. 3, the control circuit performs self-adaptation based on real-time analysis of the power PinDC(t). To this end, the control circuit 3 preferentially comprises a means of measuring the power PinDC(t), a DC voltage UpDC (t) sensor 10 and a DC current IpDC (t) sensor 11. It also comprises a processing unit 12 equipped with software and hardware means for executing functions and storing data. The data is evaluated in real time over an observation period Tobs (typically 1 ms) and updated periodically. The power PinDC is thus estimated according to the following formula:

$$PinDC ( t ) = \frac{1}{Tobs} {\int }_{Tobs} UpDC ( t ) . IpDC ( t ) . dt \qquad ( Equation 1 )$$

There is also a built-in battery 13 to power the control circuit 3. A mains power supply is also possible, optionally in combination with the built-in battery.

We will now describe the dimensioning elements and the determination of the performance variables of the TETS according to the invention.

The control circuit 3 is notably characterized by its ability to automatically modulate the voltage UpDC (t).

It has an optional output 5, preferably digital, for supplying data to the user.

It has an optional input 6, preferably digital, allowing the environment to supply it with data, in particular for self-calibration.

The dimensioning elements of the TETS are as follows.

The efficiency of the complete TETS, denoted RendTETS, is defined by:

$$RendTETS ( k , PoutDC , UpDC ) = \frac{PoutDC}{PinDC} \qquad ( Equation 2 )$$

$$0 \leq RendTETS \leq 1 \qquad ( Equation 3 )$$

The efficiency is based on losses, that is the power dissipation Pdiss in the TETS. This power dissipation in watts can be expressed based on the power PinDC and said efficiency.

$$Pdiss = PinDC ( 1 - RendTETS ) \qquad ( Equation 4 )$$

The aim of the present invention is notably to reduce the power dissipation Pdiss as much as possible, and in any case to keep Pdiss below a maximum value deemed acceptable, such that the increase in temperature in living tissue does not exceed the limits imposed by physiology and/or international standards.

Toff is the duration during which the switch IT is closed (Off phase), so the power is not transferred from the source to the load of the implant. This switch IT closes when the voltage UsDC (t) has reached the UsDCmax value, and it remains closed until it reaches the UsDCmin value. During the Off phase, the load is powered by the filter capacitor of the rectifier, whose capacitance is denoted CStock and will have been used to store energy during the charging phase (IT open). During the Off phase, PinDC(t) is substantially constant and is Poff. This power Poff is substantially dissipated as heat in the cables, the windings and the switch. This power Poff varies substantially according to the following formula wherein Rsp represents the series resistive effects of the primary circuit and Rss represents the series resistive effects of the secondary circuit. The coefficient Coeff1 depends on the design and is substantially constant regardless of k and PoutDC.

$$P i n D C ( t ) {\vert}_{i T = 1} = POff ( k , UpDC ) \approx coeff 1 \frac{UpD {C}^{2}}{R p + \frac{{k}^{2} LsLp \cdot {w}^{2}}{R s}} \qquad ( Equation 5 )$$

The duration Toff of the Off phase is substantially defined by the following formula wherein Coeff2 depends on the design and is substantially constant regardless of k and PoutDC.

$$TOff \approx \frac{Coeff 2}{P o u t D C} \qquad ( Equation 6 )$$

From the equation hereinbefore, it can be seen that the measurement of Toff makes it possible to finely estimate the value of PoutDC by applying the following formula:

$$Pout D C \approx \frac{Coeff 2}{TOff} \qquad ( Equation 7 )$$

Coeff2 is determined by the design and its value can be stored in a memory in the control circuit 3. In addition, its value can be updated/refined by exchanging data between the implant and the control circuit 3, for example during an initialization phase and/or regularly.

It is observed that POff depends on coupling k and on UpDC. It is easy to see why it is important to know the value of Toff in order to be able to optionally “deactivate” the inverter (UpDC=0 or a low value) for a duration roughly equal to Toff once this change of state has been detected. Real-time knowledge of PoutDC by exchanging data between the implant and the control circuit 3 improves the estimate of Toff. However, this deactivation is not necessary if the design has sufficiently reduced the contribution of Poff to total losses. In doing so, the effects of unacceptable electromagnetic pollution in certain constrained environments is eliminated, which is why the preferential embodiment does not implement such deactivation during the Off phase.

It is observed in the previous equation that in the absence of coupling (k=0 or less than kmini), the input power PinDC becomes very high. This power is then entirely dissipated in the external part. An absence of coupling can thus be detected and the inverter “deactivated” (UpDC=0 or a low value) for a predetermined duration, typically a waiting time of 1 s, triggering an alert to the user then reactivating it intermittently to detect coupling again when the user has realigned the primary and secondary coils.

Ton is the duration during which the switch IT is open, so the power is transferred from the external primary circuit to the implantable secondary circuit. During the On phase, when the TETS is in its correct operating range, the power absorbed by the TETS is denoted POn and is substantially constant, depending only on the coupling coefficient k and the DC voltage UpDC. It is worth noting that this power POn is also substantially independent of the power absorbed by the load PoutDC.

$$P i n D C ( t ) {\vert}_{i T = 0} \approx P O n ( k , UpDC ) \qquad ( Equation 8 )$$

The efficiency of the TETS can be expressed based on a maximum efficiency which depends on the coupling k, the power PoutDC and the DC voltage UpDC as well as the duration Ton and a design time constant denoted TEq. The following equation takes into account the highly non-linear behavior of the TETS. Thus, and according to the invention, the device will automatically modify the value of UpDC in such a way that both the theoretical maximum achievable efficiency is reached, but also that the duration Ton is as long as possible, and in any case much longer than TEq.

$$η T E T S \approx η max ( k , PoutDC , UpDC ) \cdot \frac{T o n}{( T o n + T E q )} \qquad ( Equation 9 )$$

This TEq variable depends not only on the design parameters (the output hysteresis width, the mean voltage UsDC and the storage capacity Cstock), but also on the load resistance and an equivalent internal resistance RgEq(k), itself dependent on the coupling k. It is understood that the high non-linearity of the TETS makes its optimization tricky since TEq, Ton, and the efficiency all depend on UpDC, PoutDC and k.

$$T E q \approx \frac{Δ U s D C}{U s D C} . \frac{2 \cdot RgEq ( k ) \cdot Rl}{R l + 2 \cdot RgEq} \cdot CStock \qquad ( Equation 10 )$$

The equivalent internal resistance RgEq depends substantially only on Ton, Toff, coupling k and design parameters. RdssOn is defined as representing the series resistance of the circuit of the switch IT and the following is obtained:

$$R g E q ( k , Ton , Toff ) \approx R s s + \frac{R dssOn \cdot Toff}{T o n + Toff} + \frac{R s p}{{k}^{2}} \cdot \frac{L p}{L s} \qquad ( Equation 11 )$$

As already seen, Rss represents the series resistances of the secondary circuit and Rsp represents the series resistances of the primary circuit.

It can be seen that this equivalent resistance RgEq must be minimized first and that Ton must be maximized first. In order to reduce Rss, a secondary winding with a very large copper cross-section is used as a priority, and conventionally a Litz wire in order to avoid the skin effect when the frequency Fex is high. However, according to the present invention, this resistance Rss is minimized by using a frequency Fex that is not too high (typically <300 kHz) and by using a PCB (Printed Circuit Board) secondary coil with multilayer conductors.

The contribution of the resistor RdssOn becomes very high if the switch IT is too far from the secondary coil. According to the invention, it is also planned to place the switch IT in the immediate vicinity of the secondary coil, and ideally directly on the PCB. In this preferred configuration, all of the electronics of control circuit 9 are placed on the PCB of the secondary coil. Thus, the connection between the TETS and the implant can be made via a very low-voltage DC link, considerably improving patient safety in the event of accidental insulation failure.

The coupling coefficient k can be estimated according to the following law depending on Ton, Toff and UpDC, given that Coeff3 only depends on design parameters. The importance of precisely identifying Ton and TOff, as well as UpDC is understood.

$$kestimated \approx Coeff 3 \cdot UpDC \cdot \frac{TOn \cdot TOff}{T O n + TOff} \qquad ( Equation 12 )$$

For a conventional TET 2 design, the expression for the minimum coupling coefficient to ensure the operation of the conventional TET 2 is given by the following equation wherein coeff4 again depends only on design parameters. This kmini value is the one below which the conventional TET 2 can no longer operate, due to a “stall”. In the conventional case, this kmini value does not correspond to the minimum acceptable coupling value, bearing in mind that the loss power must not be too high. This value then also depends on the power PoutDC. In the conventional case, this systematically leads to a considerable reduction in the operating range of the conventional TET 2 to include both the minimum coupling and the maximum power. Said losses depend on PoutDC and on UpDC. For a given operating point (k and PoutDC fixed), the losses decrease as UpDC decreases. It is clear that it is in our interest to decrease UpDC to reduce losses, but that this also reduces the operating range. This shows the benefits of automatically adapting the value of UpDC based on the actual operating conditions k (t) and PoutDC(t). Starting from a reference UpDC, called UpDCNominal, and a nominal coefficient kNominal, and given a maximum UpDCMax value, and minimum UpDCMin, it is understood that as long as k>kmini, the optimal value of UpDC will be less than UpDCMax. Starting from a situation for which UpDC=UpDCNominal, and k>kmini, the control circuit will automatically choose to increase or decrease UpDC in order to move towards an optimal state for which the efficiency of the TETS according to the invention is improved with respect to the conventional case. In this way, the TETS according to the invention also has the possibility when the power PoutDC is not maximum, of operating at lower coupling and thus the invention also enables the operating range of the TETS to be increased considerably.

$$kmini \approx \frac{coeff 4}{U pDC} \frac{1}{\sqrt{Ls \cdot Lp} \cdot w} \qquad ( Equation 13 )$$

In the same way, the following expression describes the stall coupling kmaxi beyond which the conventional TET 2 can no longer operate. This time it can be seen that the stall depends not only on the voltage UpDC but also on the load consumption PoutDC. Once again, and for other reasons, it can be seen that to increase the operating range (increase kmaxi), UpDC must be increased. For the same reasons as previously, this systematically leads to oversizing UpDC in cases where coupling is not maximal and thus to overconsumption. In the same way as previously, the benefits of automatically adapting UpDC are understood. Starting from a reference UpDC, it is understood that as long as k<kmaxi, the optimum value of UpDC will be less than UpDCMax. Starting from a situation for which UpDC=UpDCNominal, and k<kmaxi, the device will automatically choose to increase or decrease UpDC in order to move towards an optimal state for which the efficiency of the TETS will be improved with respect to the conventional case. This situation may change over time, even if k remains constant, based on the instantaneous value of PoutDC.

$$kmaxi \approx coeff 5 \frac{U p D C}{P o u tDC \cdot \sqrt{Ls \cdot Lp} \cdot w} \qquad ( Equation 14 )$$

According to the invention, the kmini operating range can be increased by maximizing the value of the secondary inductance Ls. To achieve this, it is useful to add a magnetic core to the secondary coil. From the formula giving the value of kmaxi, it is understood that increasing the value of Ls leads to a reduction in the operating range on the side of maximum coupling. Thus, in the case of the conventional TET 2, a compromise has been made that does not optimize the solution. The present invention eliminates this compromise by automatically adapting the value of UpDC and thus it is preferable to increase the value of the secondary inductance Ls. Preferentially, the magnetic core used is non-conductive and substantially lossless. One of the preferred solutions is the use of a superparamagnetic composite core placed in the secondary winding. The superparamagnetic filler can be placed directly in the core of the planar windings, or else in the PCB, or else in the overmolding and/or protective material of the secondary coil.

Preferentially, the secondary and primary resonance capacitors are selected such that the circuits are at resonance in the absence of coupling, that is when k=0. Thus, this leads to overconsumption on the primary side in the event of decoupling, and thus to the detection of decoupling. This also means very low consumption, almost zero on the primary side when the coils are coupled but the switch IT is closed. Thus, the single analysis of the power PinDC(t) makes it possible to automatically control the TETS according to the invention.

Optionally and without affecting the fundamental performance of the TETS according to the invention, the operating frequency of the inverter can be adapted to the resonance frequency of the secondary circuit based on information supplied by the implant during a calibration and pairing step.

According to the invention, it is planned to control the inverter preferentially with a Pulse Width Modulation (PWM) control in order to optimize the current spectrum and therefore the electromagnetic pollution of the conventional TET 2. The choice of frequency Fex is preferentially relatively low, for example less than 301 kHz, to reduce the electromagnetic pollution and the skin effect, and to allow the implementation of PWM. The frequency Fex can be chosen less than 151 kHz, or even less than 81 kHz, which corresponds to the limiting frequencies of international standards.

Two pre-calculated PWM sequences can be provided for rapid switching from one to the other, notably when decoupling is detected, or else during the OFF phase. The triggering of this total or partial deactivation is then controlled by the control circuit.

The automatic control of UpDC enables a reduction in the amplitude of the voltage at the terminals of the coils Ls and Lp, thus improving not only the efficiency but also the reliability and the safety of goods and people. Preferentially, overvoltages are kept less than 70 V peak (very low voltage limit) in order to keep any leakage currents in the event of insulation failure below a critical threshold for users.

In order to increase the efficiency of the rectifier, it is preferential to use a half-wave rectifier rather than a full-wave rectifier which would increase the diode waste voltage. This precaution is not necessary if the voltage UsDC is sufficiently large with respect to the diode waste (typically over 10 V, or even 20 V, or even 30 V, or even 50 V). In the case of half-wave rectification, a TVS (transient-voltage-suppression) protection device is added to limit the reverse voltage across the diode.

In order to reduce losses in the entire TETS transfer line, the power electronics on the primary side, notably the inverter, are positioned as close as possible to the primary coil, typically at a distance less than the diameter of the primary coil. This reduces the series resistances of the primary circuit Rsp and also the electromagnetic pollution.

A normal operating mode of the TETS will now be described according to the invention.

The graph in FIG. 4 shows a cycle with a Ton+Toff duration. It is observed that the power is systematically less than a high threshold PinDCDec and that it falls below a low threshold PinDCOff, for example 1 W.

The voltage UpDC (t) is set to a starting value, for example the nominal value UpDC=UpDCNom.

This is not a limitation of the invention; other continuation strategies are possible, in particular starting with the minimum voltage UpDCMin or else with a maximum voltage UpDCMax.

The estimated parameter Toff_est is characterized by the duration during which PinDC(t)<PinDCOff.

The parameter Ton_est is characterized by the duration during which PinDC(t)>PinDCOff.

The control circuit can then calculate the average PinDC_est value as follows

$$PinDC\_est = \frac{1}{TOn\_est + Toff\_est} {\int }_{T o {n}_{-} e s t + {Toff}_{-} e s t} P i n D C ( t ) . dt \qquad ( Equation 15 )$$

The control circuit can calculate an estimate of the power delivered by the TETS to the load, PoutDC_Est according to the following formula:

$$PoutDC\_est \approx \frac{Coeff 2}{TOff} \qquad ( Equation 16 )$$

The control circuit can therefore estimate the transfer efficiency RendTETS_est and the power dissipation Pdiss_est.

$$RendTETS\_est = \frac{PoutDC\_est}{PinDC\_est} \qquad ( Equation 17 )$$

$$Pdiss\_est = PinDC\_est ( 1 - RendTETS\_est ) \qquad ( Equation 18 )$$

And finally the control circuit can estimate the coupling coefficient k_est

$${k}_{e s t} = Coeff 3 \cdot UpDC\_est \cdot \frac{TOn\_est \cdot TOff\_est}{TOn\_est + TOff\_est} \qquad ( Equation 19 )$$

At the end of the cycle, the estimated variables are delivered to the user and analyzed then stored in memory: Ton_Est, Toff_est, PinDC_est, PoutDC_est, RendTETS_Est, Pdiss_est, k_est. Depending on the threshold, certain alarms can be triggered, etc.

Particular attention should be paid to the Ton_Est value. Indeed, if Ton_Est is too high, this means that the voltage UpDC is close to the stall value. It must then no longer be reduced. It may be decided to keep it stable for one cycle or else to increase it.

In all other cases, a priori, the value of the voltage UpDC is reduced by one DeltaUpDC increment.

At the end of the next cycle, the parameters are estimated again and the same logic is applied. In addition to the instantaneous values, the parameters are compared with their values obtained in the previous cycle and which were stored in memory. Thus, the control circuit is able to identify whether reducing the voltage UpDC by the DeltaUpDC increment has improved or degraded instantaneous transfer performance, in particular the efficiency.

The decision to increase or decrease the value of UpDC is now based not only on using real-time data, but also on its variation with respect to the previous cycle. The cycle thus repeats itself ad infinitum, guaranteeing a TETS whose operating point is automatically set to an optimum. It may be decided that the optimum performance is the minimum power PinDC, or else the maximum efficiency, or else the minimum loss Pdiss, or any other parameter estimated from the available variables.

An operating mode in the event of decoupling will now be described.

At any time, even during a cycle or in the absence of a cycle, if PinDC(t) is too high, the TETS detects a loss of coupling and can be deactivated for a predefined time, for example a few seconds. Information is given to the user. At the end of the waiting time, the TETS is reactivated from a starting cycle.

An operating mode in the event of overconsumption or underconsumption will now be described.

At the end of a cycle, if the estimated PoutDC consumption is too high (overconsumption) or too low (underconsumption), it can be decided to inform the user and either continue transferring energy, or stop the transfer. There may of course be several thresholds leading to different decisions based on the nature of the implant. An exchange of data between the implant and the device can be useful in order to make the best possible decision.

An operating mode in the event of a stall will now be described.

It is possible for the TETS to stall if, for example, the power PoutDC and/or the coupling k change abruptly, including within an acceptable operating range. In this specific case, no cycle is observed, that is Ton or Toff are too long and no power changeover is observed. This means that the switch IT is blocked, a priori in the open position, and the TETS is unable to provide sufficient power to the implant. This is detected by the use of a watchdog, which triggers an action even if the value of PinDC(t) does not change for a pre-determined duration, for example 5 s.

In this case, the voltage UpDC must be increased to allow re-connection. There are several strategies which depend on the nature of the implant:
Slow increase in UpDC
Instant switch to UpDCMax
Instant switch to UpDCNom
Etc.

An operating mode specifically taking into account a heart pump type implant will now be described.

When controlling a pulsatile heart pump, the power PoutDC(t) is not constant but is highly predictable. Indeed, in the case of a heart pump, consumption follows the heartbeat. In the case of a heart pump comprising a rechargeable battery, switching to recharge mode leads to an increase in power consumption. This information can be provided by the implant in real time to the control circuit in order to further optimize performance, for example via an electrocardiogram measurement, or else via a cardiac output measurement or any other means. In fact, this information makes it possible to anticipate changes in PoutDC(t) and thus optimize the values of UpDC other than by a blind control algorithm of UpDC. Typically, the value of UpDC will be modified upwards or downwards in larger increments, depending on the estimated actual consumption.

It should be noted, however, that this solution is optional, and that even in the event of loss of communication between the implant and the control circuit, the performance of the TETS is greatly improved with respect to a conventional TET. Thus, the feedback link from the implant to the external part is not involved in the reliability of the TETS function according to the invention.

When a storage capacitor is dimensioned as accurately as possible, which is of great interest from the point of view of size and reliability. And when the control circuit is working perfectly, the optimum voltage UpDC will follow the instantaneous consumption of the load and present a pulsatile appearance.

In these conditions, it is highly advantageous to have additional information such as the estimated instantaneous consumption in order to be able to anticipate it and speed up the adjustment of the optimum value of UpDC. This synchronization can be achieved by exchanging real-time information such as power consumption in watts, heart rate in Hz, QRS detection time, etc., between the implant and the external part of the TETS.

It follows that the control circuit takes this information into account to optimize dynamic convergence towards the minimum power.

In short, this extends the operating range of the TETS in extreme cases of PoutDC(t) and k (t), which improves the usability of the TETS.

FIG. 5 shows the evolution of the TETS operating curve according to the invention. UpDC_Opt represents the value of UpDC that maximizes the power transfer efficiency. The low limits are characterized by a stall or else prohibitive overheating.

It can be seen that the operating range according to coupling k is increased because:
It is possible to have correct operation even with a maximum coupling of “1”, thus the very concept of kmaxi is meaningless with the invention
It is possible to adapt the kmini value based on the power transferred to the load and thus extend the value of the conventional TET 2 when the actual power is less than the maximum power. Since the maximum power is always higher than the rated power, this greatly extends the usability of the TETS

It can be seen that the UpDC value that leads to optimized efficiency is largely dependent on operating conditions and that the self-adaptive solution converges towards this optimum in a certain and reliable manner. Of course, the invention is not limited to the examples disclosed above. Many modifications can be made to these examples without departing from the scope of the present invention as disclosed.

1.-18. (canceled)

19. A transcutaneous energy transmission system for powering an implant, the system comprising:
a primary circuit comprising an inverter able to be powered by a DC voltage UpDC and a direct current IpDC, and a primary coil Lp able to be powered by the inverter; and
a secondary circuit comprising a secondary coil Ls intended to be in magnetic coupling with the primary coil according to a magnetic coupling coefficient k, a secondary capacitor Cs, a rectifier able to supply the implant with a DC voltage UsDC; the secondary circuit further comprising a switch IT for powering or not powering the rectifier and a control circuit for the switch IT controlled locally from the voltage UsDC;
the system further comprises a self-adaptive control circuit, connected to the primary circuit and configured to automatically control the DC voltage UpDC from the single measurements of UpDC and IpDC, and according to a constrained optimization algorithm; the self-adaptive control circuit being further configured to estimate system performance variables from the measurements of UpDC and IpDC; and the constrained optimization algorithm being configured with at least one predetermined constraint associated with one of the system performance variables;
said system performance variables comprise at least one or more of the following instantaneous variables: a duration Toff during which the switch IT is closed, a duration Ton during which the switch IT is open, an instantaneous continuous power PinDC supplying the inverter, a continuous power PoutDC at the output of the secondary circuit, an energy transfer efficiency RendTETS, a power dissipation Pdiss in the system; and
said constraints comprise at least one of a minimum duration Toff during which the switch IT is closed, a maximum duration Ton during which the switch IT is open, a minimum instantaneous power PinDC, a maximum efficiency RendTETS, and a minimum power dissipation Pdiss.

20. The system according to claim 19, characterized in that the measurements and the estimated variables are performed cyclically.

21. The system according claim 19, characterized in that the secondary coil is made from tracks of a printed circuit PCB.

22. The system according to claim 21, characterized in that the switch IT is integrated into the printed circuit PCB of the secondary coil Ls.

23. The system according to claim 19, characterized in that a magnetic core is placed in the secondary coil Ls or in the primary coil Lp.

24. The system according to claim 23, characterized in that the magnetic core is a superparamagnetic composite core.

25. The system according to claim 19, characterized in that the primary circuit further comprises a primary capacitor Cp; the primary coil Lp and the primary capacitor Cp have values so as to be in resonance when the coupling k between the primary coil and the secondary coil is below a predetermined coupling threshold.

26. The system according to claim 19, characterized in that the secondary coil Ls and the secondary capacitor Cs have values so as to be in resonance when the coupling k between the primary coil and the secondary coil is below a predetermined coupling threshold.

27. The system according to claim 19, further including a radio link able to connect the self-adaptive control circuit with the implant so as to retrieve real-time information from the implant.

28. The system according to claim 27, characterized in that the self-adaptive control circuit is configured to adapt the operating frequency of the inverter to the resonance frequency of the secondary circuit based on information from the implant.

29. The system according to claim 19, further including at least one pulse width modulator to control the inverter at a frequency less than 301 kHz.

30. The system according to claim 19, further including a precalculated pulse width modulator with one or more harmonic contents of predefined amplitude, frequency and phase.

31. The system according to claim 19, further including a watchdog to trigger an action when the continuous instantaneous power PinDC supplying the inverter does not vary in intensity beyond a predetermined power threshold for a predetermined duration.

32. The system according to claim 19, characterized in that the self-adaptive control circuit comprises a DC voltage sensor, a direct current sensor and a processing unit.

33. A method for controlling a DC voltage UpDC in a transcutaneous energy transmission system as defined in claim 19, the method comprising, at each cycle defined by open and closed states of the switch IT, the following steps:
measuring the DC voltage UpDC and direct current IpDC;
estimating system performance variables from the measurements of UpDC and IpDC;
storing these variables; and comparing with values of these variables obtained during a previous cycle; said system performance variables comprise at least one or more of the following instantaneous variables: a duration Toff during which the switch IT is closed, a duration Ton during which the switch IT is open, an instantaneous continuous power PinDC supplying the inverter, a continuous power PoutDC at the output of the secondary circuit, an energy transfer efficiency RendTETS, a power dissipation Pdiss in the w system; controlling the DC voltage UpDC from the single measurements of UpDC and IpDC, and according to a constrained optimization algorithm configured with at least one predetermined constraint and associated with one of the system performance variables;
said constraints comprise at least one of a minimum duration Toff during which the switch IT is closed, a maximum duration Ton during which the switch IT is open, a minimum instantaneous power PinDC, a maximum efficiency RendTETS, and a minimum power dissipation Pdiss.
